(19) United States
(12) Patent Application Publication
(10) Pub. No.: US 2015/0073485 A1
(43) Pub. Date: Mar. 12, 2015

(54) SURGICAL INSTRUMENT AND METHOD

(71) Applicant: Warsaw Orthopedic, Inc., Warsaw, IN (US)

(72) Inventor: Brian A. Butler, Atoka, TN (US)

(73) Assignee: Warsaw Orthopedic, Inc., Warsaw, IN (US)

(21) Appl. No.: 14/021,784

(22) Filed: Sep. 9, 2013

(51) Int. Cl. A61B 17/88

(52) U.S. Cl. CPC A61B 17/8872
USPC 606/279; 606/86 A

(57) ABSTRACT

A surgical instrument includes a first member including a capture element and an engagement surface engageable with an implant. A second member is disposed with the first member. An actuator is engageable with the second member such that the capture element releasably engages the implant. The actuator is configured to translate the first member between a first position such that the implant is movable relative to the first member and a second position such that the first member is fixed with the implant. Systems and methods of use are disclosed.

### TECHNICAL FIELD

The present disclosure generally relates to medical devices for the treatment of musculoskeletal disorders, and more particularly to a surgical system for implant delivery to a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods and bone fasteners can be delivered to a surgical site. This disclosure describes an improvement over these prior art technologies.

### SUMMARY

In one embodiment, a surgical instrument is provided. The surgical instrument includes a first member including a capture element and an engagement surface engageable with an implant. A second member is disposed with the first member. An actuator is engageable with the second member such that the capture element releasably engages the implant. The actuator is configured to translate the first member between a first position such that the implant is movable relative to the first member and a second position such that the first member is fixed with the implant. In some embodiments, systems and methods of use are disclosed.

### BRIEF DESCRIPTION OF THE DRAWINGS

The present disclosure will become more readily apparent from the specific description accompanied by the following drawings, in which:

FIG. 1 is a side view of components of one embodiment of a system in accordance with the principles of the present disclosure;

FIG. 2 is a side view of the components shown in FIG. 1;

FIG. 3 is side view of the components shown in FIG. 1;

FIG. 4 is a perspective view of the components shown in FIG. 1;

FIG. 5 is a cross section view of the components shown in FIG. 3;

FIG. 6 is a cross section view of the components shown in FIG. 2;

FIG. 7 is a break away view of the components shown in FIG. 1;

FIG. 8 is a break away view of the components shown in FIG. 5;

FIG. 9 is a perspective view of a component of one embodiment of a system in accordance with the principles of the present disclosure;

FIG. 10 is a break away view of the components shown in FIG. 2;

FIG. 11 is a break away view of components of one embodiment of a system in accordance with the principles of the present disclosure;

FIG. 12 is a perspective view of the components shown in FIG. 11;

FIG. 13 is a cross section view of the components shown in FIG. 12;

FIG. 14 is a break away view of the components shown in FIG. 12;

FIG. 15 is a perspective view of components of one embodiment of a system in accordance with the principles of the present disclosure;

FIG. 16 is a break away view of the components shown in FIG. 15; and

FIG. 17 is a cross section view of components of one embodiment of a system in accordance with the principles of the present disclosure.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine.

In one embodiment, the surgical system and method includes an instrument for percutaneous insertion and rotation of spinal rods. In one embodiment, the surgical system and method include a pre-bent spinal rod inserted into a surgical site. In one embodiment, the spinal rod is rotated after insertion such that the bend in the spinal rod is disposed along a particular plane to achieve a correction to the spine. In one embodiment, the surgical system includes an instrument that can hold the spinal rod and rotate the spinal rod at the surgical site. In one embodiment, the surgical system includes an instrument that includes a spring configured to release and engage a clamp about a spinal rod. In one embodiment, the surgical system includes a wire is configured to fix the spinal rod with the instrument.

In one embodiment, the surgical system includes an actuator that is configured to rotate to open a clamp for insertion of a spinal rod. In one embodiment, the surgical system includes a spinal rod inserted into an instrument until the spinal rod snaps into a clamp. In one embodiment, the surgical system includes an actuator configured for rotation to lock a clamp around a spinal rod. In one embodiment, the surgical system includes a tab configured to lock and unlock a gear mechanism disposed with the instrument to facilitate or prevent rotation of a spinal rod.

In one embodiment, the surgical system includes an instrument configured for attachment to a rod for minimally invasive surgery. In one embodiment, the surgical system can rotate the rod to facilitate insertion for a derotation correction maneuver. In one embodiment, the surgical system includes an outer sleeve that actuates a clamp to hold the rod, and an inner shaft that locks and/or unlocks a ratchet mechanism to rotate a spinal rod. In one embodiment, the surgical system includes a wire to hold a spinal rod in place. In one embodiment, the surgical system can be used as an inserter and a rotator in, for example, a percutaneous lateral fusion.

In one embodiment, one or all of the components of the system are disposable, peel-pack, pre-packed sterile devices used with an implant. One or all of the components of the system may be reusable. The system may be configured as a kit with multiple sized and configured components.

In one embodiment, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system and methods may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

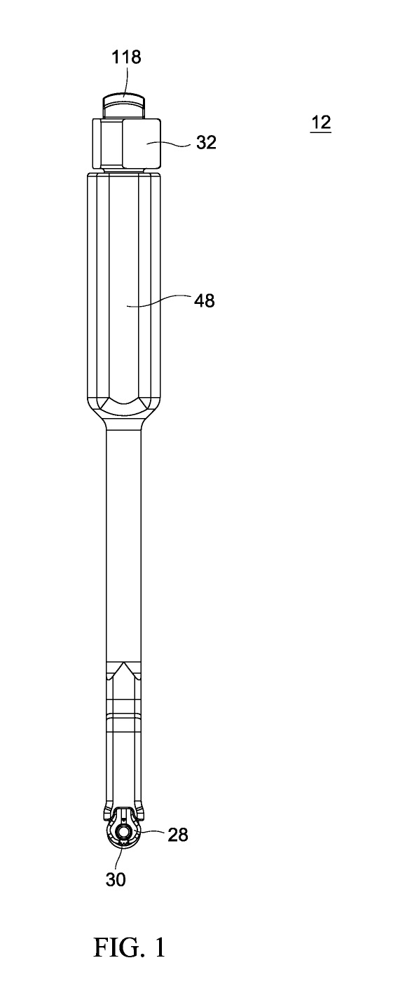

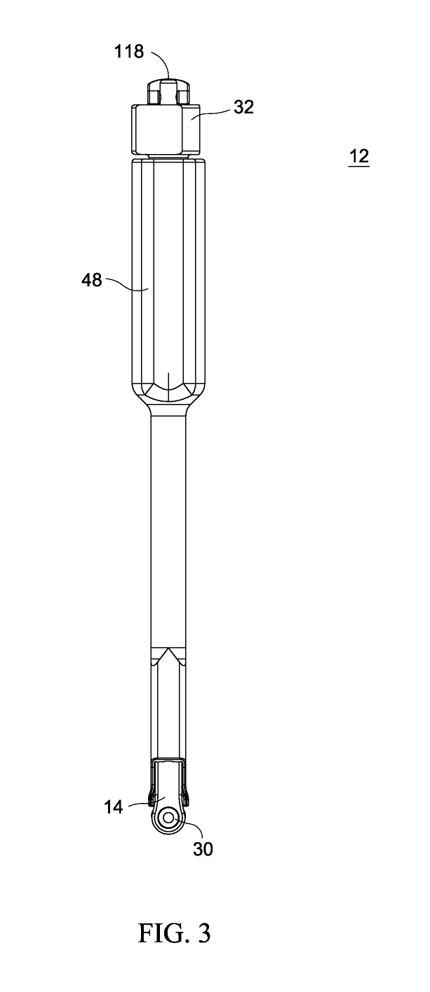

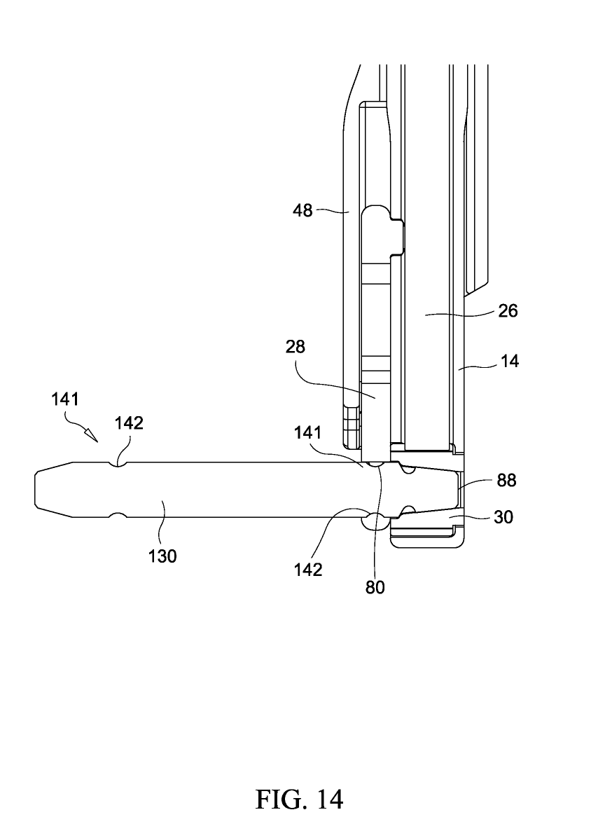

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-14, there are illustrated components of a surgical system, such as, for example, a surgical system 10 in accordance with the principles of the present disclosure.

The components of system 10 can be fabricated from biologically acceptable materials suitable for medical applications, including metals, synthetic polymers, ceramics and bone material and/or their composites, depending on the particular application and/or preference of a medical practitioner. For example, the components of system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is employed, for example, with a minimally invasive procedure, including percutaneous techniques, mini-open and open surgical techniques to deliver and introduce an implant, such as, for example, a spinal rod, at a surgical site within a body of a patient, for example, a section of a spine. In one embodiment, system 10 may deliver and introduce a spinal rod for a derotation correction treatment. In one embodiment, system 10 may insert and rotate a spinal rod in a percutaneous lateral fusion procedure.

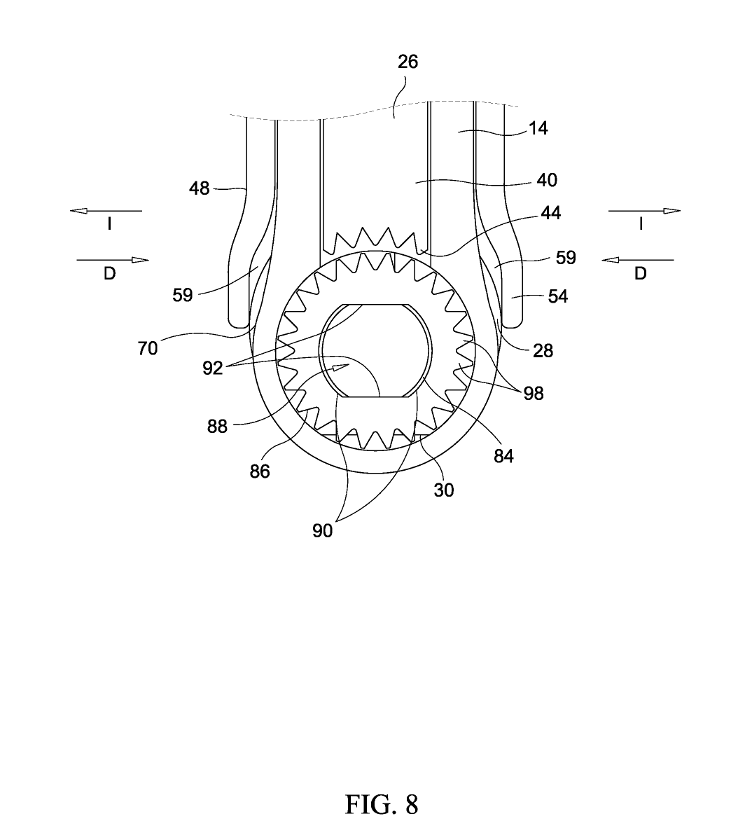

System 10 includes a surgical instrument 12 that is configured for engagement with a spinal construct, such as, for example, a spinal rod 130, as shown in FIG. 8 and discussed herein. Instrument 12 includes a first member, such as, for example, an inner sleeve 14. Sleeve 14 extends between an end 16 and an end 18 and defines a longitudinal axis L1. Sleeve 14 includes an outer surface 20 and an inner surface 22. A portion of surface 20 is configured to form a cavity 58 configured for disposal of a capture element 28, as discussed herein. Surface 22 defines a passageway 24 configured for translation of a shaft 26, as discussed herein. End 18 of sleeve 14 includes capture element 28 and an engagement surface engageable with an implant, such as, for example, spinal rod 130. In one embodiment, the engagement surface comprises a rotator 30, as discussed herein, which engages spinal rod 130. In one embodiment, the engagement surface comprises a distal end of shaft 26 that directly engages and/or contacts an outer surface of spinal rod 130 between a locked position and a non-locking position, similar to that described herein with regard to rotator 30. End 16 is configured for engagement with an actuator 32, as discussed herein. In one embodiment, end 16 includes a threaded portion 34 configured to engage actuator 32. In some embodiments, the first member and/or engagement surface, as described herein, is engageable with the implant, as described herein, in one or a plurality of positions, such as, for example, a first, a second and a third position.

In some embodiments, all or only a portion of surfaces 20, 22 may have alternate surface configurations, such as, for example, rough, threaded for connection with surgical instruments, arcuate, undulating, porous, semi-porous, dimpled, polished and/or textured. In some embodiments, sleeve 14 is circular in shape but may have alternate cross section configurations, such as, for example, oval, oblong, triangular, rectangular, square, polygonal, irregular, uniform, non-uniform, variable and/or tapered.

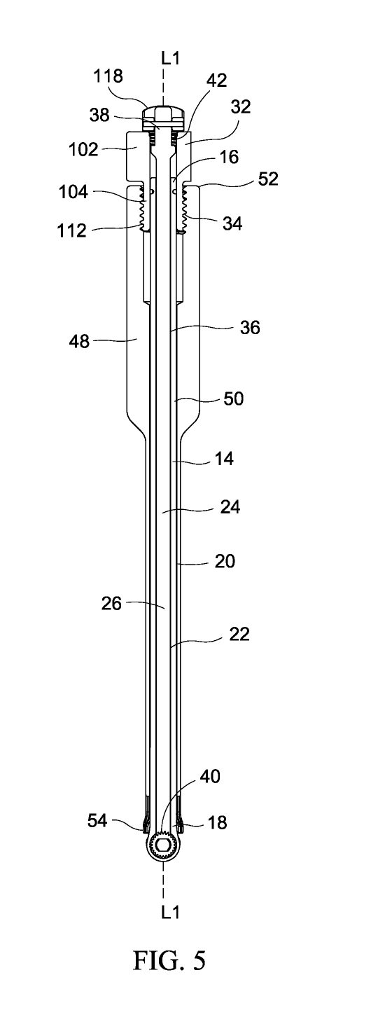

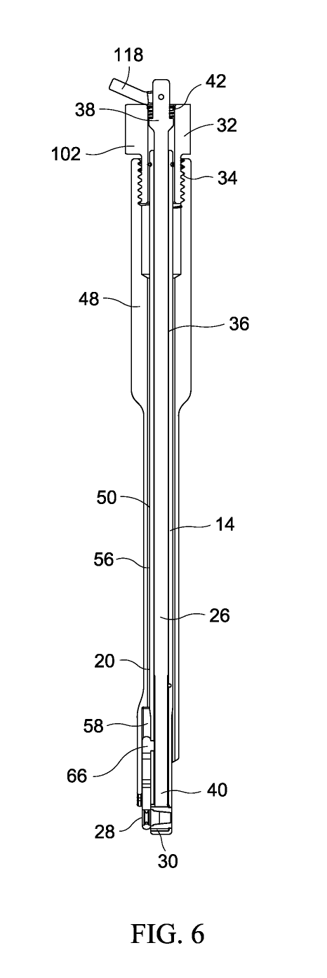

Shaft 26 is configured for disposal in passageway 24 such that shaft 26 axially translates to engage and disengage from rotator 30, as discussed herein. Shaft 26 includes an outer surface 36 and extends between an end 38 and an end 40. End 38 is configured for connection with actuator 32. As shown in FIG. 6, in one embodiment, shaft 26 is resiliently biased, such as, for example, with a spring 42 into engagement with rotator 30. Spring 42 is configured to bias shaft 26 into engagement with rotator 30 between a locked position, as shown in FIG. 5 and a non-locking position, as shown in FIG. 8. In an expanded, non-compressed configuration, spring 42 biases shaft 26 into engagement with rotator 30. Actuation of spring 42 causes shaft 26 to disengage from rotator 30. In one embodiment, in the expanded, non-compressed configuration, spring 42 can be biased such that shaft 26 is disengaged from rotator 30. End 40 includes at least one mating surface, such as, for example, a plurality of gear teeth 44 configured for engagement with at least one mating surface of rotator 30, as discussed herein.

In some embodiments, all or only a portion of surface 36 may have alternate surface configurations, such as, for example, rough, threaded for connection with surgical instruments, arcuate, undulating, porous, semi-porous, dimpled, polished and/or textured. In some embodiments, shaft 26 is circular in shape but may have alternate cross section configurations, such as, for example, oval, oblong, triangular, rectangular, square, polygonal, irregular, uniform, non-uniform, variable and/or tapered.

Instrument 12 includes a second member, such as, for example an outer sleeve 48. Sleeve 48 includes an inner surface 50 and extends along axis L1 between an end 52 and an end 54. Surface 50 defines a cavity, such as, for example, a passageway 56 configured for moveable disposal of sleeve 14. A portion of surface 50 and a portion of surface 20 define cavity 58 configured for moveable disposal of capture element 28. Cavity 58 includes concave end surfaces 59 extending from end 54. Cavity 58 facilitates axial translation of capture element 28 such that proximal translation causes capture element 28 to move inwardly to lock spinal rod 130 and distal translation causes capture element 28 to move outwardly to unlock spinal rod 130, as discussed herein.

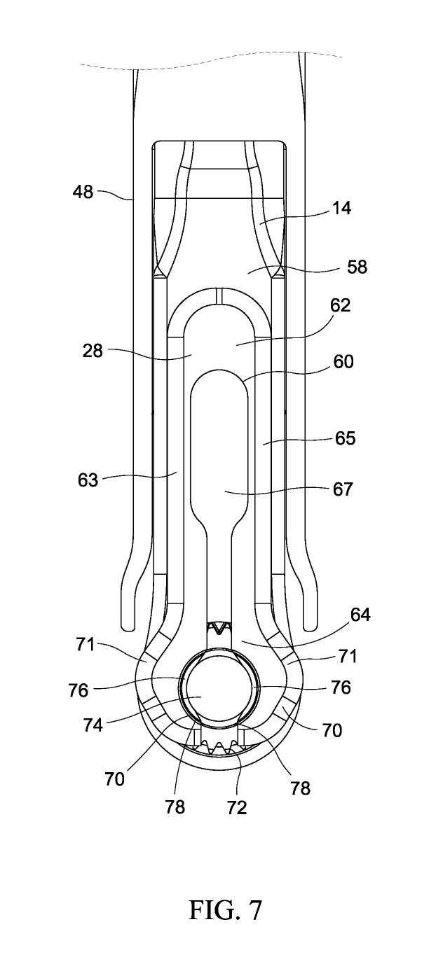

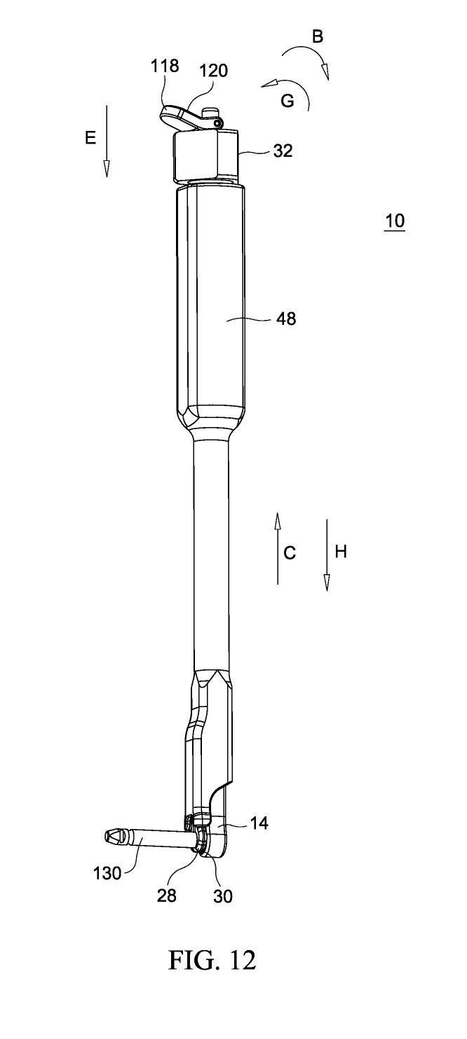

As shown in FIG. 7, capture element 28 includes an elongate portion 60 extending between an end 62 and an end 64. Capture element 28 extends along surface 20 within cavity 58. End 62 includes a protrusion, such as, for example, a pin 66 configured to engage an opening 68 disposed in surface 20. Pin 66 is configured to fix capture element 28 with sleeve 14. In one embodiment, as shown in FIG. 7, portion 60 includes an extension 63 and an extension 65. Extensions 63, 65 extend in parallel relation and form a cavity 67 therebetween. In some embodiments, extensions 63, 65 can extend in alternate configurations such as, for example, transverse, perpendicular and/or other angular orientations such as acute or obtuse and/or co-axial. Cavity 67 facilitates inward and outward movement of extensions 63, 65, as discussed herein. Each extension 63, 65 includes an arcuate end portion forming a capture portion, such as, for example, jaws 70. Jaws 70 are moveable between a non-locking orientation, as shown in FIG. 7, and a locking orientation, as shown in FIG. 12. As shown in FIG. 7, jaws 70 are arcuate in shape such that concave portions 72 are configured to face each other forming a circular cavity 74. Cavity 74 is configured for disposal of spinal rod 130. Each of jaws 70 includes an inner surface 76. Surfaces 76 define a capture mating surface 78 configured to engage a capture mating surface 141 of spinal rod 130, as discussed herein.

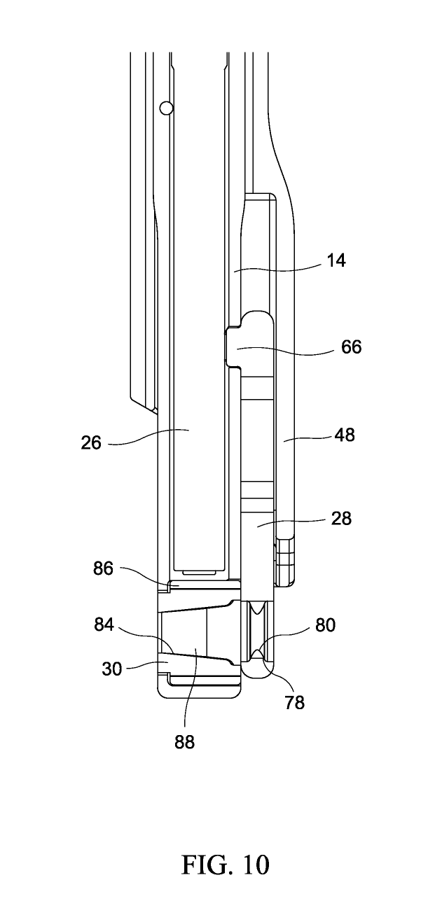

Extensions 63, 65 are resiliently biased such that in the non-locking position, extensions 63, 65 and jaws 70 are positioned outwardly from each other to facilitate insertion of spinal rod 130. In the locking position, extensions 63, 65 and jaws 70 are positioned inwardly towards each other to facilitate capture of spinal rod 130. Translation of capture element 28 proximally into sleeve 48 causes convex surfaces 71 of jaws 70 to translate along concave end surfaces 59 such that jaws 70 are moved towards each other into the locking orientation to lock spinal rod 130 with capture element 28 and instrument 12. As shown in FIG. 10, mating surface 78 includes a circumferential flange 80 configured to engage a circumferential recess 142 of spinal rod 130. Capture of spinal rod 130 facilitates engagement with rotator 30 and movement of spinal rod 130 to a surgical site.

In some embodiments, jaws 70 may have alternate cross section configurations, such as, for example, oval, oblong, triangular, rectangular, square, polygonal, irregular, uniform, non-uniform, variable and/or tapered. In some embodiments, all or only a portion of surface 76 may have alternate surface configurations, such as, for example, rough, threaded for connection with surgical instruments, arcuate, undulating, porous, semi-porous, dimpled, polished and/or textured.

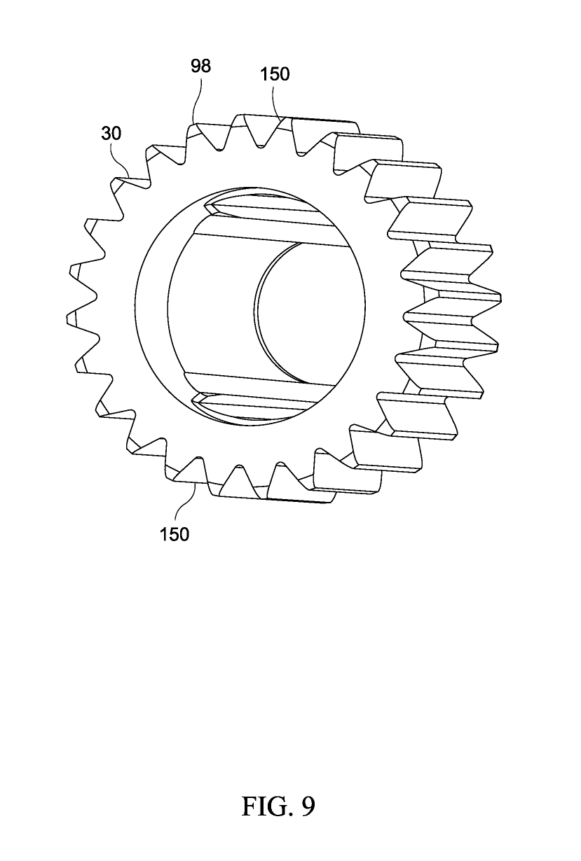

Rotator 30 includes an inner surface 84 and an outer surface 86, as shown in FIG. 8. In one embodiment, rotator 30 includes a circular shape. Surface 84 defines a cavity 88 that is tapered along its depth to facilitate insertion and capture of spinal rod 130. In some embodiments, cavity 88 can be non-tapered, such as, for example, having a uniform, constant dimension or diameter. Surface 84 includes substantially concave sidewalls 90 and substantially planar top and bottom walls 92 configured to engage a portion of spinal rod 130. In one embodiment, as shown in FIG. 9, surface 84 includes at least one wire 150 to lock spinal rod 130 with rotator 30. In some embodiments, wire 150 can be made from stainless steel alloys, commercially pure titanium, titanium alloys, Grade 5 titanium, super-elastic titanium alloys, cobalt-chrome alloys, stainless steel alloys, superelastic metallic alloys such as Nitinol.

Surface 86 includes at least one mating surface, such as, for example, a plurality of gear teeth 98 configured for engagement with gear teeth 44. Teeth 98 and teeth 44 engage to lock rotator 30 to resist and/or prevent rotator 30 and spinal rod 130 from relatively rotating. Disengagement of teeth 98 and teeth 44 allows rotator 30 to freely rotate relative to sleeve 14. In one embodiment, teeth 44 and teeth 98 can include a ratchet mechanism for incremental rotation. In some embodiments, rotator 30, cavity 88 and/or surface 84 may have alternate cross section configurations along the depth of rotator 30, such as, for example, cylindrical, oval, oblong, triangular, rectangular, square, hexagonal including for example hexalobular, polygonal, irregular, uniform, non-uniform, non-tapered, constant dimension, and/or variable. In some embodiments, end 40, surface 86 and/or the mating surfaces described herein may comprise alternate mating surface configurations, such as, for example, friction fit, pressure fit, pin-in-groove, keyed connection, slotted connection, fasteners, rough, threaded, arcuate, undulating, dimpled and/or textured.

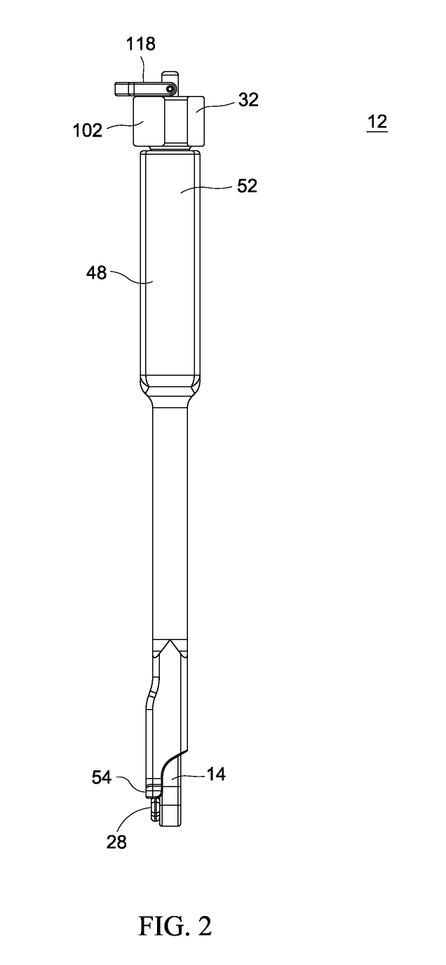

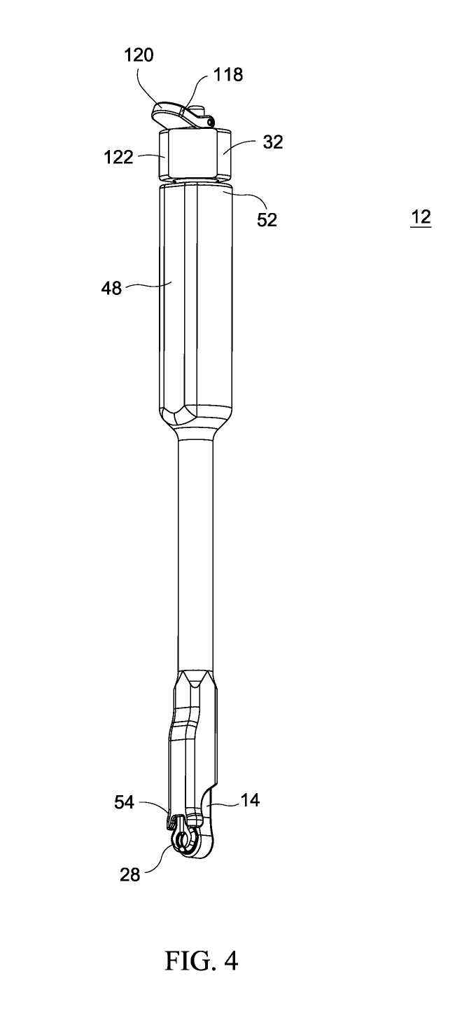

Actuator 32 extends along axis L1 and includes a rotatable portion 102 and an elongated portion 104. In some embodiments, actuator 32 may extend from sleeves 14, 48 in alternate configurations such as, for example, transverse, perpendicular and/or other angular orientations such as acute or obtuse, co-axial and/or parallel. Portion 104 is configured to extend in a cavity 106 formed by sleeve 14 and sleeve 48 at ends 16, 52. Portion 104 includes an inner surface 108 that defines a cavity 110 configured to receive end 16 of sleeve 14. Surface 108 includes a threaded portion 112 configured to engage portion 34 of sleeve 14 to facilitate axial translation of sleeve 14, which causes pivoting of capture element 28 via engagement with sleeve 48. Portion 102 is rotatable about axis L1 such that rotation of portion 102 causes threaded portions 112, 34 to axially translate sleeve 14. Portion 102 includes a surface 114 that defines a cavity 116 configured for disposal of spring 42. In one embodiment, actuator 32 includes a lever 118 configured to engage spring 42 to bias shaft 26 between the locked position and the non-locking position with rotator 30. Lever 118 is depressible between a first configuration, as shown in FIG. 4, and second configuration, as shown in FIG. 2, to actuate spring 42.

Actuator 32 includes an outer surface 122 configured as a gripping surface. In some embodiments, all or only a portion of surface 122 may have alternate surface configurations, such as, for example, rough, threaded for connection with surgical instruments, arcuate, undulating, porous, semi-porous, dimpled, polished and/or textured.

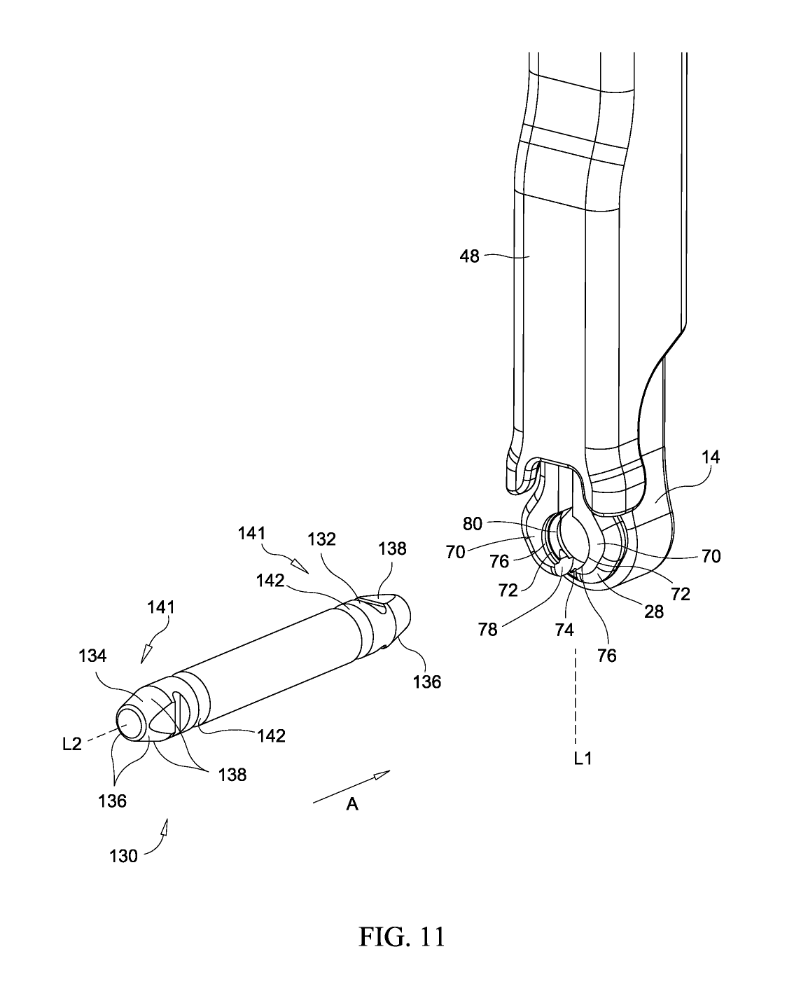

System 10 includes a spinal construct, such as, for example, spinal rod 130 that extends between an end 132 and an end 134 along a longitudinal axis L2, as shown in FIG. 11. Ends 132, 134 each have a tapered portion configured for mating engagement with the tapered portion of rotator 30. Each of ends 132, 134 includes convex side portions 136 and planar top and bottom portions 138 configured for engagement with rotator 30. Each of ends 132, 134 includes a capture element mating portion 141, such as, for example, a circumferential recess 142 configured for engagement with flange 80 of jaws 70 to facilitate capture of spinal rod 130. In some embodiments, spinal rod 130 includes a single recess 142 disposed with end 132 or end 134. Spinal rod 130 is configured for alignment and insertion with instrument 12. In some embodiments, spinal rod 130 may be inserted into instrument 12 in alternate configurations such as, for example, perpendicular, transverse or other angular orientations such as acute or obtuse, co-axial and/or parallel.

In operation, to capture and deliver spinal rod 130 to a surgical site, spinal rod 130 is positioned substantially orthogonal to instrument 12 along axis L2, as shown by arrow A in FIG. 11. Jaws 70 are disposed in an open position such that jaws 70 are disengaged from concave end surfaces 59, as shown in FIG. 11. An end 132 or 134 of spinal rod 130 is positioned such that convex side portions 136 engage concave sidewalls 90 and planar top and bottom portions 138 engage planar top and bottom walls 92 of rotator 30, as shown in FIG. 12. Recess 142 mates with flange 80 such that spinal rod 130 mates in a fixed configuration with jaws 70.

Portion 102 of actuator 32 is rotated, such as, for example, in a clockwise direction, as shown by arrow B in FIG. 12, to lock jaws 70 with spinal rod 130. Rotation of actuator 32 causes sleeve 14 to translate, in the direction shown by arrow C in FIG. 12, causing portion 60 to translate within cavity 58. Translation of portion 60 causes jaws 70 to move inwardly along concave end surfaces 59, in the direction shown by arrow D in FIG. 8, to a locked position. Translation along concave end surfaces 59 causes jaws 70 to move inwardly to tighten and lock spinal rod 130 with instrument 12.

Engagement of spinal rod 130 with rotator 30 prevents spinal rod 130 from rotating relative to rotator 30. In an initial position, as shown in FIG. 5, rotator 30 is locked with shaft 26 for delivering and insertion of instrument 12 adjacent to a surgical site. Lever 118 is disposed in a first position such that spring 42 is in an expanded configuration such that spring 42 biases shaft 26 into a locked configuration with rotator 30. Teeth 44 engage teeth 98 such that rotation of rotator 32 and spinal rod 130 attached therewith is resisted and/or prevented relative to shaft 26 and instrument 12. As such, the orientation of spinal rod 130 can be adjusted and manipulated with instrument 12. In some embodiments, rotator 30 is locked with shaft 26 such that rotation of instrument 12 facilitates rotation of spinal rod 130 into engagement with one or a plurality of fasteners, such as, bone screws disposed with vertebrae, for example, rotating instrument 12 rotates spinal rod 130 within implant cavities of the screws for delivery, insertion and/or positioning of spinal rod 130. In some embodiments, instrument 12 rotates spinal rod 130 in a clockwise direction and/or a counter-clockwise direction.

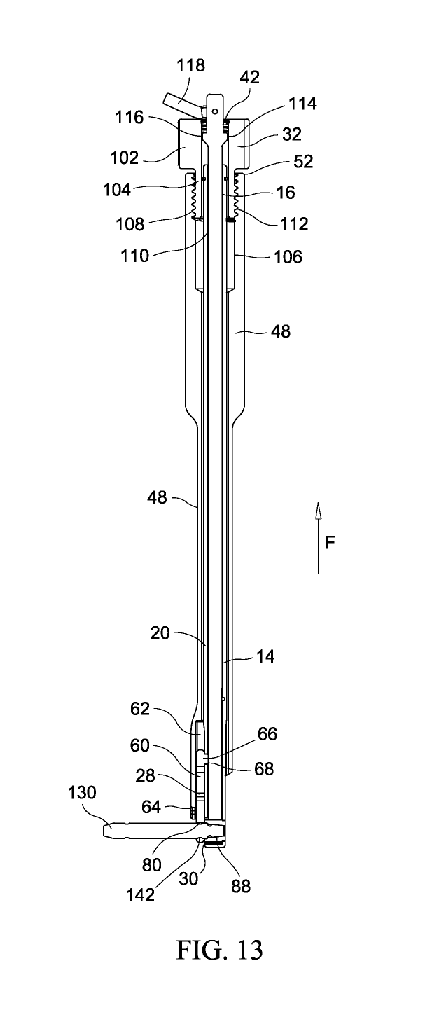

To facilitate rotation of rotator 30 relative to shaft 26 and instrument 12, lever 118 is depressed, in the direction shown by arrow E in FIG. 12, to actuate spring 42. Lever 118 engages spring 42 to axially translate shaft 26, in the direction shown by arrow F in FIG. 13, to disengage teeth 44 from teeth 98. In this configuration, rotator 30 and spinal rod 130 attached therewith can rotate relative to sleeves 14, 48 and instrument 12 to facilitate movement of instrument 12 and spinal rod 130 at the surgical site. This configuration facilitates manipulation and adjustment of the orientation of sleeves 14, 48 and/or instrument 12 without altering the position and orientation of spinal rod 130. In some embodiments, instrument 12 is rotated relative to rotator 30 and spinal rod 130 to facilitate selective orientation of instrument 12. In some embodiments, instrument 12 rotates relative to spinal rod 130 in a clockwise direction and/or a counter-clockwise direction.

To disengage spinal rod 130, portion 102 of actuator 32 is rotated, such as, for example, in a counter-clockwise direction, as shown by arrow G in FIG. 12, to dispose jaws 70 in a non-locking orientation and release spinal rod 130 from instrument 12. Rotation of actuator 32 causes sleeve 14 to translate, in the direction shown by arrow H in FIG. 12, causing portion 60 to translate within cavity 58. Translation of portion 60 cause jaws 70 to move outwardly along concave end surfaces 59, in the direction shown by arrows I in FIG. 8, to a non-locking orientation. Translation along concave end surfaces 59 cause jaws 70 to move outwardly to release spinal rod 130.

In assembly, operation and use, as shown in FIGS. 11-12, system 10, similar to that described above, is employed with a surgical procedure for treatment of a spinal disorder affecting a section of a spine of a patient, as discussed herein. System 10 may also be employed with other surgical procedures. For example, system 10 can be used with a surgical procedure for treatment of a condition or injury of an affected section of the spine including vertebrae (not shown).

In use, to treat the affected section of vertebrae, a medical practitioner obtains access to a surgical site including vertebrae in any appropriate manner, such as through incision and retraction of tissues. In some embodiments, system 10 may be used in any existing surgical method or technique including open surgery, mini-open surgery, minimally invasive surgery, including percutaneous surgical implantation, whereby vertebrae are accessed through a micro-incision, or sleeve that provides a protected passageway to the area. Once access to the surgical site is obtained, the particular surgical procedure is performed for treating the spinal disorder. System 10 is employed to augment the surgical treatment. System 10 can be delivered or implanted as a pre-assembled device or can be assembled in situ. One or all of the components of system 10 may be completely or partially revised, removed or replaced during or after the surgical procedure.

Pilot holes or the like are made in vertebrae for receiving the shaft of a bone fastener (not shown). The components of system 10 are disposed adjacent vertebrae at a surgical site and the bone fasteners of system 10 are manipulable to fix or otherwise connect spinal rod 130 to vertebrae. In one embodiment, extenders (not shown) are employed to support the bone fasteners and provide a pathway for connecting spinal rods 130 with the bone fasteners. A driver (not shown) may be employed with the extenders to fix the bone fasteners with vertebrae.

Upon fixation of the bone fasteners with vertebrae, spinal rod 130 is positioned substantially orthogonal to instrument 12. Jaws 70 are disposed in an open orientation, as described herein, such that jaws 70 are disengaged from concave end surfaces 59. An end 132 or 134 of spinal rod 130 is positioned with instrument 12 such that convex side portions 136 engage concave sidewalls 90 and planar top and bottom portions 138 engage planar top and bottom walls 92 of rotator 30. Recess 142 mates with flange 80 such that spinal rod 130 mates in a fixed configuration with jaws 70.

Portion 102 of actuator 32 is rotated in a clockwise direction to lock jaws 70 with spinal rod 130. Rotation of actuator 32 causes jaws 70 to move inwardly to tighten and lock spinal rod 130 with instrument 12, as described herein.

Lever 118 is disposed in a first position and rotator 30 is locked with shaft 26, as described herein, for delivering and insertion of instrument 12 adjacent to the surgical site. As such, the orientation of spinal rod 130 can be adjusted and manipulated with instrument 12, as described herein. To facilitate rotation of shaft 26 and instrument 12 relative to rotator 30 and spinal rod 130, lever 118 is depressed to actuate spring 42 and disengage teeth 44 from teeth 98, as described herein. In this configuration, rotator 30 and spinal rod 130 attached therewith can rotate relative to sleeves 14, 48 and instrument 12 to facilitate movement of instrument 12 and spinal rod 130 at the surgical site. This configuration facilitates manipulation and adjustment of the orientation of sleeves 14, 48 and/or instrument 12 without altering the position and orientation of spinal rod 130.

Actuator 32 is rotated in a counter-clockwise direction to dispose jaws 70 in a non-locking orientation and release spinal rod 130 from instrument 12, as described herein. Upon completion of a procedure, described herein, the surgical instruments, assemblies and non-implanted components of spinal correction system 10 are removed and the incisions are closed.

One or more of the components of system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of system 10. In some embodiments, system 10 may comprise implants, which include one or a plurality of plates, connectors, longitudinal elements and/or bone fasteners for use with a single vertebral level or a plurality of vertebral levels.

In some embodiments, system 10 includes an agent, which may be disposed, packed, coated or layered within, on or about the components and/or surfaces of system 10. In some embodiments, the agent may include bone growth promoting material, such as, for example, bone graft to enhance fixation of the components and/or surfaces of system 10 with vertebrae. In some embodiments, the agent may include one or a plurality of therapeutic agents and/or pharmacological agents for release, including sustained release, to treat, for example, pain, inflammation and degeneration.

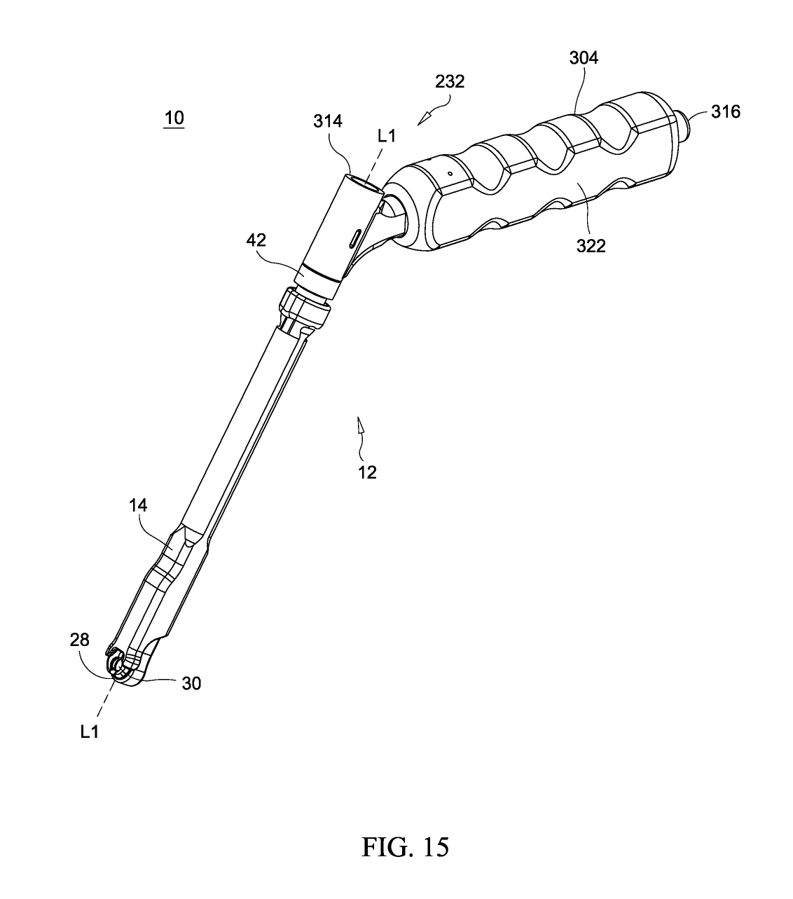

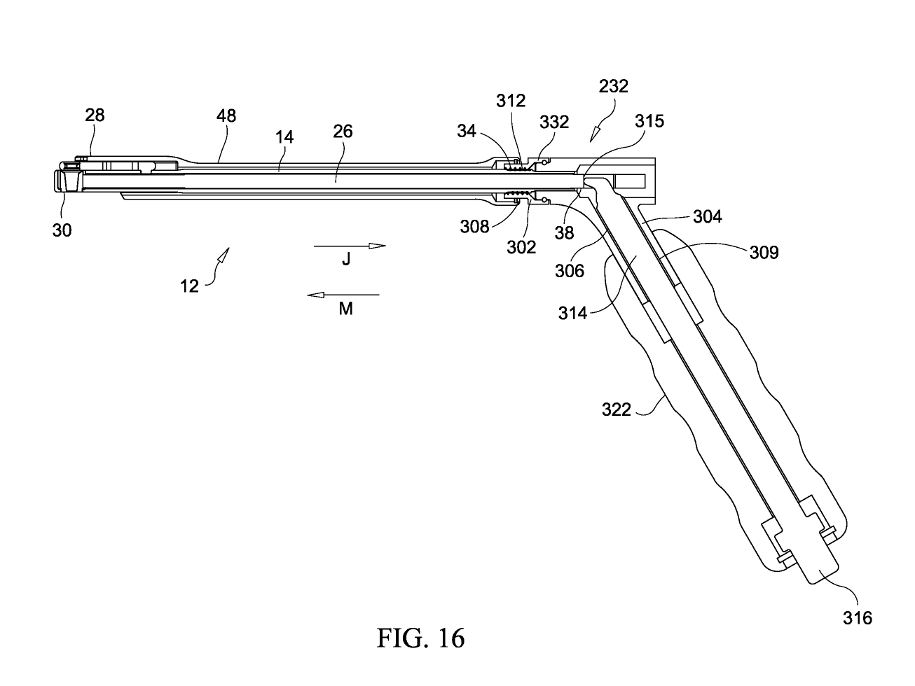

In one embodiment, as shown in FIGS. 15 and 16, system 10 includes instrument 12, described with regard to FIGS. 1-14, which comprises an actuator 232 extending transverse to axis L1 and includes a rotatable portion 302 and a handle portion 304. Portion 302 includes an inner surface 308 that defines a cavity 310 configured to receive end 16 of sleeve 14. Surface 308 includes a threaded portion 312 that engages portion 34 of sleeve 14 for disassembly and/or removal of components to facilitate, for example, cleaning. Sleeve 48 is manually translated, relative to sleeve 14, to facilitate movement of capture element 28. Sleeve 48 is biased by a spring 332 between a locked position and an unlocked position relative to capture element 28.

Handle portion 304 includes an inner surface 306 that defines a cavity 309. Cavity 309 is configured for disposal of a lever 314 that is configured to engage and disengage shaft 26 from rotator 30. Lever 314 is configured for disposal between a first locked position and a second non-locking position. Lever 314 includes an engagement surface 315 configured to engage shaft 26. Translation of lever 314 causes surface 315 to engage end 38 of shaft 26 to translate shaft 26 such that teeth 44 engage teeth 98. Portion 304 includes a button 316 configured to actuate translation of lever 314.

Actuator 232 includes an outer surface 322 configured as a gripping surface. In some embodiments, all or only a portion of surface 322 may have alternate surface configurations, such as, for example, rough, threaded for connection with surgical instruments, arcuate, undulating, porous, semi-porous, dimpled, polished and/or textured.

In operation, to capture and deliver spinal rod 130 to a surgical site, spinal rod 130 is positioned substantially orthogonal to instrument 12. Sleeve 48 is manually translated, in the direction shown by arrow J in FIG. 16, such that jaws 70 are disposed in an open orientation such that jaws 70 are disengaged from concave end surfaces 59 and sleeve 48 is in the unlocked position. Sleeve 48 is released and spring 232 biases sleeve 48, in the direction shown by arrow M in FIG. 16, to lock jaws 70 with spinal rod 130. Recess 142 mates with flange 80 such that spinal rod 130 mates in a fixed configuration with jaws 70.

Engagement of spinal rod 130 with rotator 30 resists and/or prevents spinal rod 130 from rotating relative to rotator 30. In an initial position, rotator 30 is locked with shaft 26 for delivery and insertion of instrument 12 adjacent to a surgical site. In the locked orientation, teeth 44 are engaged with teeth 98 such that rotation of rotator 32 and spinal rod 130 attached therewith is prevented relative to shaft 26 and instrument 12.

To facilitate rotation of rotator 30 relative to shaft 26 and instrument 12, button 316 is depressed to actuate translation of lever 314, in the direction shown by arrow J in FIG. 16, to translate shaft 26 to disengage teeth 44 from teeth 98. In this configuration, rotator 30 and spinal rod 130 attached therewith can rotate relative to sleeves 14, 48 and instrument 12 to facilitate movement of instrument 12 and spinal rod 130 at the surgical site. To disengage spinal rod 130, sleeve 48 is translated to release jaws 70 from spinal rod 130.

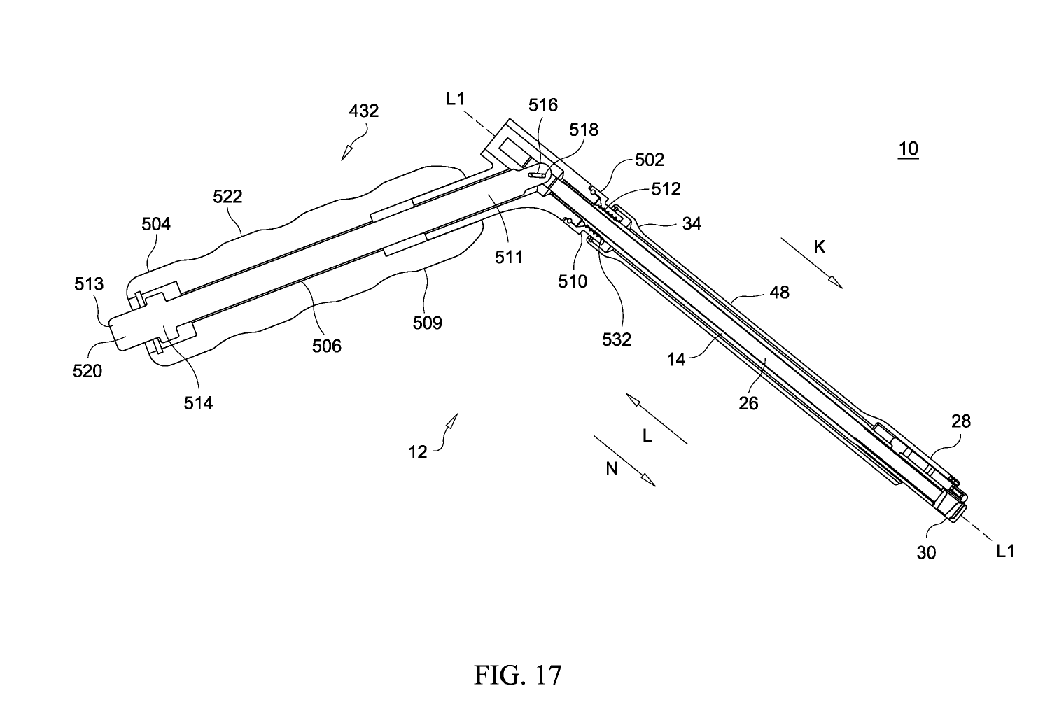

In one embodiment, as shown in FIG. 17, system 10 includes instrument 12 described with regard to FIGS. 1-14, which comprises an actuator 432 extending transverse to axis L1 and includes a rotatable portion 502 and a handle portion 504. Portion 502 includes an inner surface 508 that defines a cavity 510 configured to receive end 16 of sleeve 14. Surface 508 includes a threaded portion 512 configured to engage portion 34 for disassembly and/or removal of components to facilitate, for example, cleaning. Sleeve 48 is manually translated, relative to sleeve 14, to facilitate movement of capture element 28. Sleeve 48 is biased by a spring 532 between a locked position and an unlocked position relative to capture element 28.

Handle portion 504 includes an inner surface 506 that defines a cavity 509. Cavity 509 is configured for disposal of a lever 514 that is configured to engage and disengage shaft 26 from rotator 30. Lever 514 is configured for disposal between a first locked position and a second non-locking position. Lever 514 extends between an end 511 and an end 513. End 511 includes an aperture 516 configured to receive a pin 518 disposed with shaft 26. Translation of lever 514 within cavity 509 causes shaft 26 to move about pin 518 such that teeth 44 engage teeth 98. End 513 includes a button 520 configured to actuate translation of lever 514.

Actuator 432 includes an outer surface 522 configured as a gripping surface. In some embodiments, all or only a portion of surface 522 may have alternate surface configurations, such as, for example, rough, threaded for connection with surgical instruments, arcuate, undulating, porous, semi-porous, dimpled, polished and/or textured.

In operation, to capture and deliver spinal rod 130 to a surgical site, spinal rod 130 is positioned substantially orthogonal to instrument 12. Sleeve 48 is translated, in the direction shown by arrow L in FIG. 17, against spring 532 such that jaws 70 are disposed in an open position such that jaws 70 engage concave end surfaces 59 and sleeve 48 is in the unlocked position. To lock jaws 70 with spinal rod 130, sleeve 48 is biased by spring 532 in the opposite direction, in the direction shown by arrow N in FIG. 17, to lock sleeve 48 such that jaws 70 engage spinal rod 130. Recess 142 mates with flange 80 such that spinal rod 130 mates into a fixed configuration with jaws 70.

Engagement of spinal rod 130 with rotator 30 prevents rod from freely rotating within rotator 30. In an initial position, rotator 30 is locked with shaft 26 for insertion of instrument 12 into the surgical site. In the locked position, button 520 is depressed such that lever 514 pivots about pin 518 and moves pin 518, in the direction shown by arrow K in FIG. 17, such that shaft 26 translates, in the direction shown by arrow K, and teeth 44 are engaged with teeth 98 such that rotation of rotator 32 and spinal rod 130 attached therewith is resisted and/or prevented relative to shaft 26 and instrument 12.

To facilitate rotation of rotator 30 relative to shaft 26 and instrument 12, button 520 depressed to move lever 514 about pin 518 in the opposite direction, as shown by arrow L in FIG. 17, such that shaft 26 disengages teeth 44 from teeth 98. In this configuration, rotator 30 and spinal rod 130 attached therewith can rotate relative to sleeves 14, 48 and instrument 12 to facilitate movement of instrument 12 and spinal rod 130. To disengage spinal rod 130, sleeve 48 is translated to release jaws 70 from spinal rod 130.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first member including a capture element and an engagement surface engageable with an implant;
a second member disposed with the first member; and
an actuator being engageable with the second member such that the capture element releasably engages the implant,
the actuator being configured to translate the first member between a first position such that the implant is movable relative to the first member and a second position such that the first member is fixed with the implant to resist movement of the implant relative to the first member.

2. A surgical instrument as recited in claim 1, wherein the engagement surface comprises a rotator disposed about the implant.

3. A surgical instrument as recited in claim 2, wherein the first member includes a shaft that engages the rotator in the second position.

4. A surgical instrument as recited in claim 3, wherein the shaft includes a mating surface configured to engage a mating surface of the rotator.

5. A surgical instrument as recited in claim 1, wherein the capture element includes jaws moveable between a non-locking orientation and a locking orientation.

6. A surgical instrument as recited in claim 1, wherein the engagement surface comprises a rotator that includes an inner surface that defines a tapered cavity.

7. A surgical instrument as recited in claim 1, wherein the engagement surface comprises a rotator that includes an inner surface that defines a non-tapered, hexagonal cavity.

8. A surgical instrument as recited in claim 1, wherein the engagement surface comprises a rotator that includes an inner surface that defines a mating surface configured to engage a mating surface of the implant.

9. A surgical instrument as recited in claim 1, wherein the first member is resiliently biased to the second position.

10. A surgical instrument as recited in claim 1, wherein the actuator is rotatable for axial translation of the first member relative to the second member.

11. A surgical instrument as recited in claim 1, wherein the actuator includes a lever configured to translate the first member.

12. A method for treating a spine, the method comprising the steps of:
fastening at least one fastener with vertebrae;
providing a surgical instrument comprising a lock and a member including an engagement surface engageable with a spinal rod;
locking the surgical instrument with the spinal rod via the lock to manipulate the spinal rod into engagement with the at least one fastener wherein
the member is disposable in a first position such that the spinal rod is rotatable relative to the member and a second position such that the member is fixed with the spinal rod.

13. A method as recited in claim 12, wherein in the first position the member is translated to space the engagement surface from the spinal rod such that the spinal rod is rotatable relative to the member.

14. A method as recited in claim 12, wherein in the first position the spinal rod is rotatable relative to the member in a clockwise direction and a counter-clockwise direction.

15. A method as recited in claim 12, wherein in the second position the member and the spinal rod are rotatable in a clockwise direction and a counter-clockwise direction.

16. A method as recited in claim 12, further comprising the step of translating the member between the first position and the second position.

17. A surgical instrument comprising:
a lock engageable with an implant; and
a member including an engagement surface engageable with the implant,
the member being disposable in a first position such that the implant is rotatable relative to the member and a second position such that the first member is fixed with the implant.

18. A surgical instrument as recited in claim 17, wherein the engagement surface comprises a rotator disposed about the implant.

19. A surgical instrument as recited in claim 18, wherein the member includes a shaft that engages the rotator in the second position.

20. A surgical instrument as recited in claim 19, wherein the shaft includes a mating surface configured to engage a mating surface of the rotator.
